(19) United States
(12) Patent Application Publication
(10) Pub. No.: US 2006/0292246 A1
(43) Pub. Date: Dec. 28, 2006

(54) Characteristic mass spectral fingerprint setting method and rapid identification method for Chinese herbal medicines and prescriptions

(76) Inventors: Rey-Yuh Wu, Xizhi City (TW); Yuh-Shan Chung, Xizhi City (TW); Ya-Wen Kuo, Xizhi City (TW)

Correspondence Address: Ladas & Parry, 26 West 61st Street, New York, NY, 10023, US

(21) Appl. No.: 11/471,240

(22) Filed: Jun. 20, 2006

(30) Foreign Application Priority Data
Jun. 20, 2005 (TW) 094120472

(51) Int. Cl. G01N 33/00; A61K 36/18

(52) U.S. Cl. 424/725; 436/86

(57) ABSTRACT

A characteristic mass spectral fingerprint setting method and a rapid identification method for Chinese herbal medicines or prescriptions are provided, which comprise directly injecting extracts of Chinese herbal medicines or prescriptions into a mass spectrometer with an electrospray ionization interface via an autosampler of a liquid chromatograph without going through a separation column to make a mass spectrum analysis; performing a series of mass spectrum analyses for various Chinese herbal medicines or prescription extracts; picking out the mass spectrum peaks representing the features of each of the Chinese herbal medicines or prescriptions as respective characteristic mass spectral fingerprints to establish a characteristic mass spectral fingerprint database of the Chinese herbal medicine or prescription; directly injecting an extract of the Chinese herbal medicines or prescription to be tested into a mass spectrometer with an electrospray ionization interface to make a mass spectrum analysis; and comparing and analyzing the obtained mass spectrum data with the characteristic mass spectral fingerprint database so as to rapidly identify the Chinese herbal medicine or prescription to be tested.

### BACKGROUND OF THE INVENTION

1. Field of the Invention

The present invention relates to a characteristic mass spectral fingerprint setting method and a rapid identification method for Chinese herbal medicines or prescriptions, wherein a liquid chromatograph in combination with a high resolution and high sensitivity mass spectrometer with an electrospray ionization (ESI) device is used to rapidly identify the Chinese herbal medicine or prescription.

2. Description of the Prior Art

Many studies have been made on identification and quality control of Chinese herbal medicines (a single plant) or Chinese herbal prescriptions (a mixture of various plants) for a long time. General methods for identifying Chinese herbal medicine include appearance description, microscopic identification, and chemical qualitative tests. Common methods used in chemical qualitative tests include thin layer chromatography (TLC), spectrophotometer, gas chromatography (GC), gas chromatography-mass spectrum (MS), liquid chromatography (LC), and liquid chromatography-mass spectrum, wherein thin layer chromatography and spectrophotometer methods are poor in resolution and sensitivity. Moreover, although gas chromatography, gas chromatography-mass spectrum, liquid chromatography, and liquid chromatography-mass spectrum have preferable resolution and sensitivity, the analysis time is made longer to provide a good separating effect in the chromatographic spectrum. More importantly, Chinese herbal medicine extracts often contain substances consisting of various ingredients which have a wide range of polarity, and retention of these substances often occurs in the separation column, which may affect the performance of the separation column, or may disturb analysis results. Furthermore, there is a failure to exhibit good repeatability due to the drift phenomenon generated by retention time in separation. Therefore, the above-mentioned methods can't achieve a rapid and accurate identification for Chinese herbal medicines or prescriptions. In recent years, there have been many reports on the application of GC/MS and LC/MS in identification and quantification of analytes; however, no rapid and accurate mass spectrum analysis method for Chinese herbal medicines or prescriptions has been proposed in conventional references.

### SUMMARY OF THE INVENTION

The main purpose of the present invention is to provide a rapid identification method for Chinese herbal medicines or prescriptions to overcome the problems of rapid and accurate mass spectrum analytic effect that conventional high performance liquid chromatograph (HPLC), GC/MS, and LC/MS analytic methods cannot achieve.

Another purpose of the present invention is to provide a characteristic mass spectral fingerprint setting method for Chinese herbal medicines or prescriptions.

Another purpose of the present invention is to provide a characteristic mass spectral fingerprint database, which is constructed by integrating extracts of different solvents with characteristic mass spectral fingerprints.

### BRIEF DESCRIPTION OF THE DRAWINGS

FIG. 1 shows the ESI-MS spectra of a Radix Saposhnikoviae extract sample of embodiment 1 of the invention which is analyzed repeatedly and continuously 3 times, indicated as (a), (b), and (c) in order.

FIG. 2 shows the ESI-MS spectra of a Jade-screen Powder extract sample of embodiment 2 of the invention which is repeatedly and continuously analyzed 3 times, indicated as (a), (b), and (c) in order.

FIG. 3 shows the ESI-MS spectra of the same Rhizoma Chuanxiong extract sample of embodiment 3 of the invention which is analyzed 3 times at an interval of 7 days, indicated as (a), (b), and (c) in order.

FIG. 4 shows the ESI-MS spectra of the same Radix Angelicae Sinensis extract sample of embodiment 4 of the invention which is analyzed 3 times at an interval of 7 days, indicated as (a), (b), and (c) in order.

FIG. 5 shows the ESI-MS spectra of the same Rhizoma Chuanxiong extract sample of embodiment 5 of the invention, which is determined by using a mass spectrometer of the same type at different times and different determination sites, wherein (a) indicates the determination at site A, (b) indicates the determination of the sample of (a) after placing it at 4° C. for 1 month at site A, and (c) indicates the determination of the same sample as that of (b) at site B.

FIG. 6 shows the ESI-MS spectra of the same Radix Rehmanniae Preparata extract sample of embodiment 6 of the invention, which is determined by using a mass spectrometer of the same type at different times and different determination sites, wherein (a) indicates the determination at site A, (b) indicates the determination of a Radix Rehmanniae Preparata extract sample of (a) after placing it at 4° C. for 50 days at site A, and (c) indicates the determination of the same sample as that of (b) at site B.

FIG. 7 shows the ESI-MS spectra of Si Wu Tang prescription extract sample of embodiment 7 of the invention comprising individual extracts of the four single medicines of Si Wu Tang and the mixture of individual extracts of the same volume, wherein (a) is a Rhizoma Chuanxiong extract, (b) is a Radix Rehmanniae Preparata extract, (c) is a Radix Paeoniae Alba extract, (d) is a Radix Angelicae Sinensis extract, and (e) is a sample obtained by mixing (a), (b), (c), and (d) of the same volume.

FIG. 8 shows the ESI-MS spectra of Si Wu Tang prescription extract sample of embodiment 8 of the invention comprising individual extracts of the four single medicines of Si Wu Tang and the mixture of individual extracts of the same volume and the sample obtained by mixing individual medicines of the same weight followed by extracting with water, wherein (a) is a Rhizoma Chuanxiong water extract, (b) is a Radix Rehmanniae Preparata water extract, (c) is a Radix Paeoniae Alba water extract, (d) is a Radix Angelicae Sinensis water extract, and (e) is a sample obtained by mixing (a), (b), (c), and (d) of the same volume, and (f) is a sample obtained by mixing individual medicines of the same weight followed by extracting with water.

FIG. 9 shows the ESI-MS spectra of the same samples as Embodiment 8 by placing them at 4° C. for 14 days followed by performing the same mass spectrum analysis.

FIG. 10 shows the ESI-MS spectra of the diethyl ether extracts of Astragalus Membranaceus from different places of origin of embodiment 10 of the invention determined at different times, wherein (a) is an Astragalus Membranaceus extract from Shanxi, (b) is an Astragalus Membranaceus extract from Neimeng, (c) is a sample of (a), placed at 4° C. for 14 days, followed by ESI-MS analysis, and (d) is a sample of (b), placed at 4° C. for 14 days, followed by ESI-MS analysis.

### DETAILED DESCRIPTION

The characteristic mass spectral fingerprint setting method for Chinese herbal medicines or prescriptions according to the invention mainly comprises: with an extract obtained by extracting a Chinese herbal medicine or prescription with a solvent as an analysis sample, directly injecting the analysis sample via an autosampler of a liquid chromatograph and pushing a menstruum through a hydraulic pump. As the solvent outlet of the hydraulic pump is directly connected to an inlet of a mass spectrometer with an electrospray ionization interface, the analysis sample can be directly fed into the mass spectrometer with an electrospray ionization interface without going through a chromatographic separation column to perform a mass spectrum analysis. Thus, the mass spectrum data of the analysis sample is obtained, and then analyzed. Taking the mass spectrum peaks with certain mass spectrum intensity and representing the features of the analysis sample as the selecting standard, the characteristic mass spectral fingerprint of the analysis sample is selected.

The rapid identification method for Chinese herbal medicines or prescriptions according to the invention comprises performing a series of mass spectrum analyses for various Chinese herbal medicine or prescription extracts by the above-mentioned method; picking out the mass spectrum peaks representing the features of each Chinese herbal medicine or prescription as a respective characteristic mass spectral fingerprint; studying the obtained characteristic mass spectral fingerprints to establish a characteristic mass spectral fingerprint database of the Chinese herbal medicine or prescription; and directly injecting an extract of the Chinese herbal medicine or prescription to be tested into a mass spectrometer with an electrospray ionization interface without going through a chromatographic separation column to perform mass spectrum analysis; and comparing and analyzing the obtained mass spectrum data with the characteristic mass spectral fingerprint database, so as to rapidly identify the Chinese herbal medicine or prescription to be tested.

As the extracts in the present method haven't been separated through a chromatographic separation column, the data determined by the mass spectrometer will cover the spectra of all substances in the extracts. The representative mass spectrum peaks are chosen as the extract's characteristic mass spectral fingerprint to establish a characteristic mass spectral fingerprint database. By establishing the database, the drift phenomenon and repeatability problem in conventional methods caused by the overlong analysis time needed for the extract separation through the separation column can be efficiently avoided, so as to rapidly and accurately identify the extract to be tested.

In the present invention, a suitable liquid chromatograph can be a high performance liquid chromatograph; a suitable mass spectrometer can be a mass spectrometer with an electrospray ionization interface; a suitable solvent is from a variety of solvents that can be used in the extraction process, which include, for example, but are not limited to, water, methanol, or ethanol; a suitable Chinese herbal medicine is, for example, but is not limited to, Radix Saposhnikoviae ( [symbol] ), milkvetch root [symbol] Rhizoma Atractylodis Macrocephalae [symbol] Rhizoma Chuanxiong [symbol] Radix Rehmanniae Preparata [symbol] Radix Paeoniae Alba [symbol] Radix Angelicae Sinensis [symbol] Astragalus Membranaceus from Shanxi [symbol] or Astragalus mongholicus [symbol] a suitable prescription is, for example, but is not limited to, Jade-screen Powder [symbol] and Si Wu Tang

[symbol]

The following examples will further illustrate the characteristic mass spectral fingerprint setting method and the rapid identification method for Chinese herbal medicines or prescriptions according to the invention, but are not intended to limit the application scope of the present invention. All modifications and changes made by anyone skilled in the art, without departing from the spirit of the invention, are within the scope of the invention.

Materials:

1. Chinese herbal medicine: Radix Saposhnikoviae, milkvetch root [symbol] Rhizoma Atractylodis Macrocephalae, Rhizoma Chuanxiong, Radix Rehmanniae Preparata, Radix Paeoniae Alba, Radix Angelicae Sinensis, Astragalus Membranaceus from Shanxi and Astragalus mongholicus are commercially available from a Chinese herbal medicine importer [symbol] Directly use without further treatment.

2. Prescription: Jade-screen Powder and Si Wu Tang are commercially available from a Chinese herbal medicine importer (SUN TEN). Directly use without further treatment.

3. Methanol (Reagent-grade): Commercially available from Merck. Directly use without further treatment.

4. Deionized water.

5. Diethyl ether: Commercially available from Merck. Directly use without further treatment.

Instruments:

1. Mass spectrometer: Quadruple time of flight mass spectrometer, Z-Spray nanoflow electrospray, Harvard syringe pump, Massylynx software.

2. Hydraulic pump: Waters Alliance 2695 HPLC Pump.

3. Sample injector: Waters Alliance 2695 Autosampler.

4. Centrifuge: GS-15R, Beckman.

Preparation of the sample:

1. Extract with methanol:

Grind the Chinese herbal medicine with a grinding machine and then add 1 g of said medicine to a 15 ml centrifuge tube containing 10 ml methanol. After extracting the mixture by rotating and stirring for 24 h, put it in a centrifuge at 3000 rmp for 20 min at 4° C. and collect the supernatant for determination.

2. Extract with water:

Grind the Chinese herbal medicine with a grinding machine and then add 1 g of said medicine to a 50 ml round-bottom flask containing 25 ml deionized water. Soak for 1 h after uniformly mixing and then heat to reflux for 1 h. After cooling, put it in a centrifuge at 3000 rmp for 20 min at 4° C. and collect the supernatant for determination.

3. Extract with diethyl ether:

Grind the Chinese herbal medicine with a grinding machine and then add 1 g of said medicine to a 15 ml centrifuge tube containing 10 ml diethyl ether. After extracting the mixture by rotating and stirring for 24 h, put it in a centrifuge at 3000 rmp for 20 min at 4° C. and collect the supernatant for determination.

4. Mix extract

Uniformly mix the equivalent volume (200 μl) of extracts for determination.

ESI-MS analysis:

1. Mobile phase: 100% MeOH.

2. Flowing Rate: 0.2 ml/min.

3. Injection volume of the sample: 10 μl.

4. ESI-MS conditions

| | |
|---|---|
| category | T of MS |
| Ion Mode | ES Mode |
| Polarity | Positive |
| Start Mass | 50.0 |
| End Mass | 900.0 |
| Start Time (min) | 0.0 |
| End Time (min) | 3.0 |
| Scan Time (sec) | 1.0 |
| InterScan Time (sec) | 0.1 |
| Scans To Sum | 1000000 |
| Use Tune Page Sample Cone | YES |

### Embodiment 1

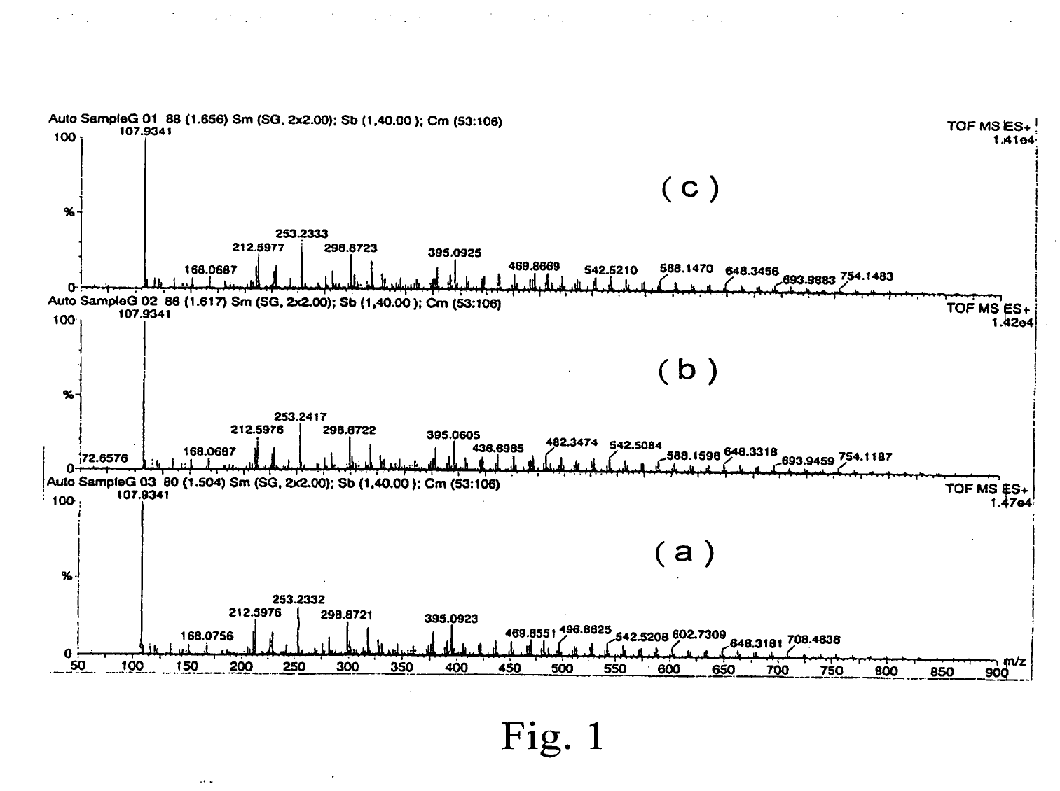

The present embodiment relates to treating a Radix Saposhnikoviae sample by using the methanol extract method of the above-mentioned preparation of sample 1 to obtain a Radix Saposhnikoviae extract; then performing a mass spectrum analysis with the above ESI-MS analytic method, in which, after each analysis, the rate of mobile phase is kept continuous to carry out the next analysis with an interval of 2 min, 3 times in total. The obtained spectra are shown in FIGS. 1(a), (b), and (c). The main mass spectrum peaks (m/z) in FIG. 1(a) are 107, 168, 212, 253, 298, 395, 436, 469, 496, 542, 588, 602, 693, 708, and 754, respectively. The spectra of FIGS. 1(b) and 1(c) are almost the same as that of FIG. 1(a).

### Embodiment 2

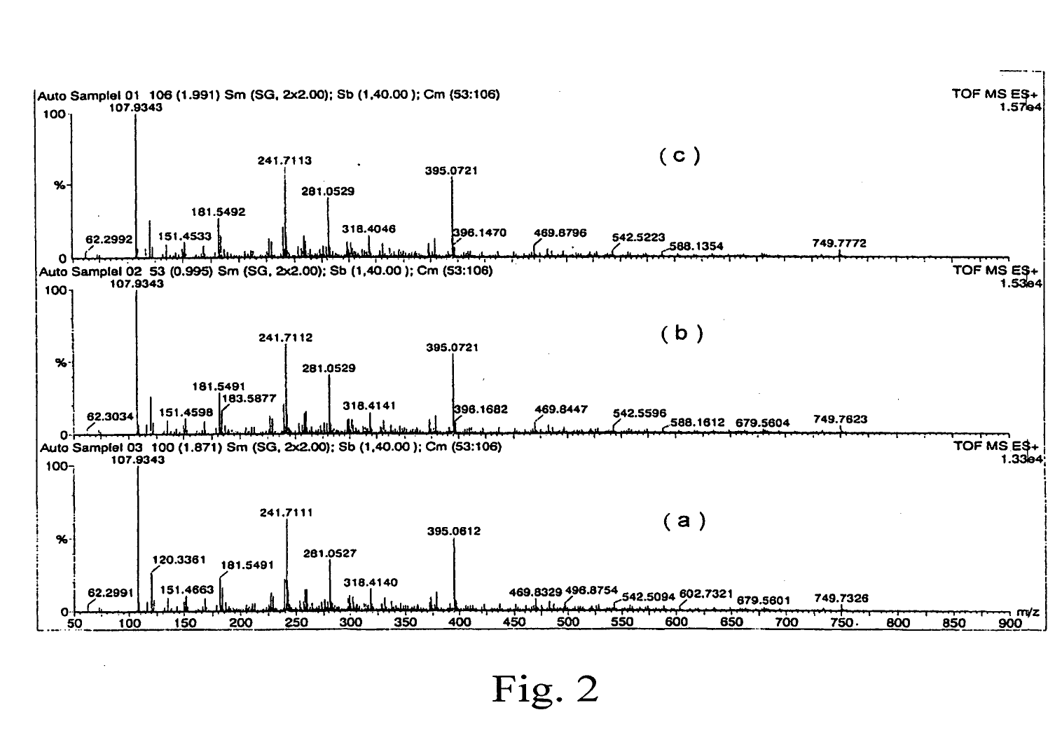

The present embodiment relates to a Jade-screen Powder sample, and the experimental steps are analogous to Embodiment 1. The obtained spectra are shown in FIGS. 2(a), (b), and (c). The main mass spectrum peaks (m/z) in FIG. 2(a) are 107, 120, 151, 181, 241, 281, 318, 395, 469, 496, 542, 588, 602, 679, and 749, respectively. The spectra of FIGS. 2(b) and 2(c) are almost the same as that of FIG. 2(a).

### Embodiment 3

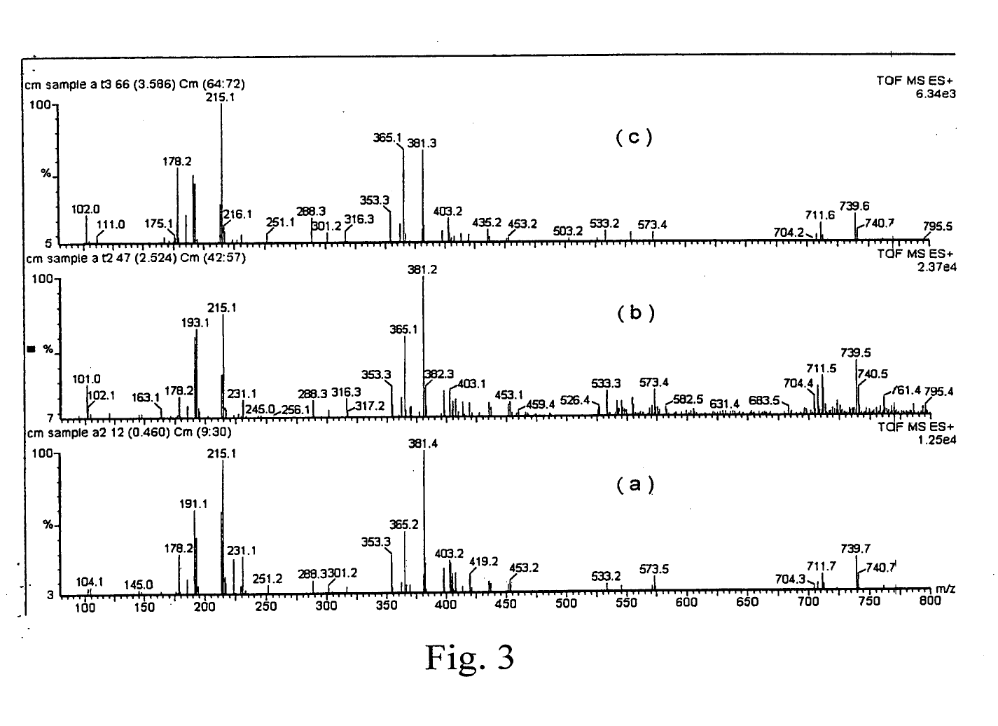

The present embodiment relates to treating a Rhizoma Chuanxiong sample by using the methanol extract method of the above-mentioned preparation of sample 1 to obtain a Rhizoma Chuanxiong extract; then performing a mass spectrum analysis with the above ESI-MS analytic method; placing the extract at 4° C. after analyzing, taking out and performing the second mass spectrum analysis 7 days later; placing the extract at 4° C. again after analyzing, taking out and performing the third mass spectrum analysis again 7 days later. The obtained spectra are shown in FIGS. 3(a), (b), and (c). The main mass spectrum peaks (m/z) in FIG. 3(a) are 178, 191, 193, 214, 215, 231, 288, 353, 365, 381, 403, 419, 453, 533, 573, 711, 739, and 740, respectively. The main mass spectrum peaks in FIG. 3(b) are almost the same as those of FIG. 3(a), but the relative density of the mass spectrum peaks is somewhat different, such as m/z=178, 191, 193. It is also the same for FIG. 3(c).

### Embodiment 4

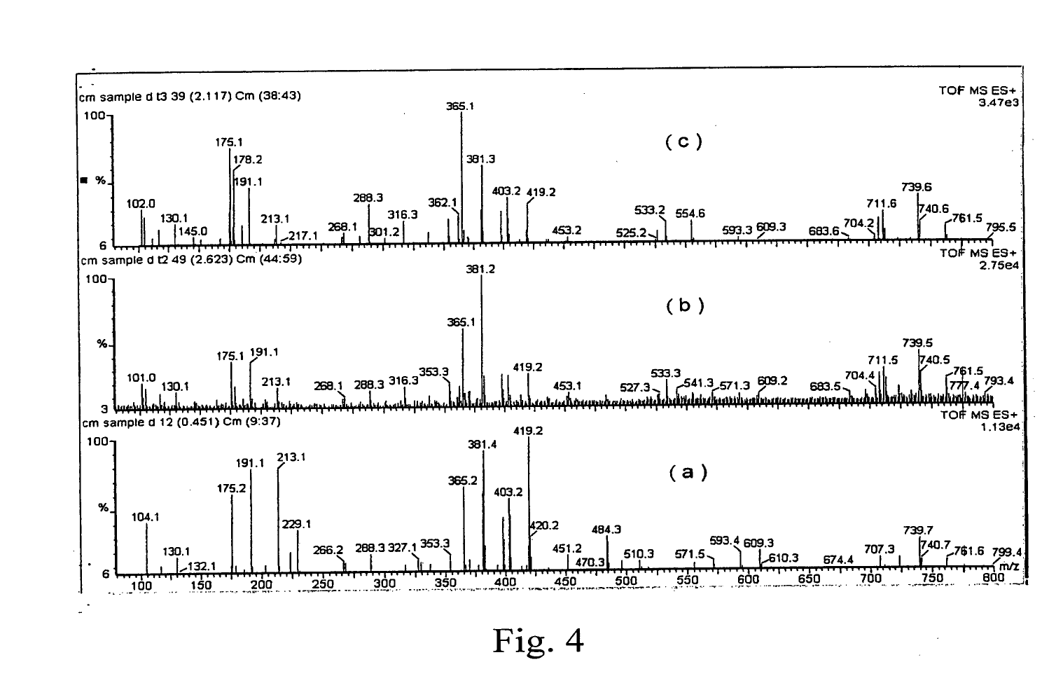

The present embodiment relates to a Radix Angelicae Sinensis sample, and the experimental steps are analogous to Embodiment 3. The obtained spectra are shown in FIGS. 4(a), (b), and (c). The main mass spectrum peaks (m/z) in FIG. 4(a) are 130, 175, 191, 213, 268, 288, 353, 365, 381, 403, 419, 593, 739, and 761, respectively. The main mass spectrum peaks in FIG. 4(b) are almost the same as those of FIG. 4(a), but the relative density of the mass spectrum peaks is somewhat different, such as m/z=381, 419. It is also the same in FIG. 4(c).

### Embodiment 5

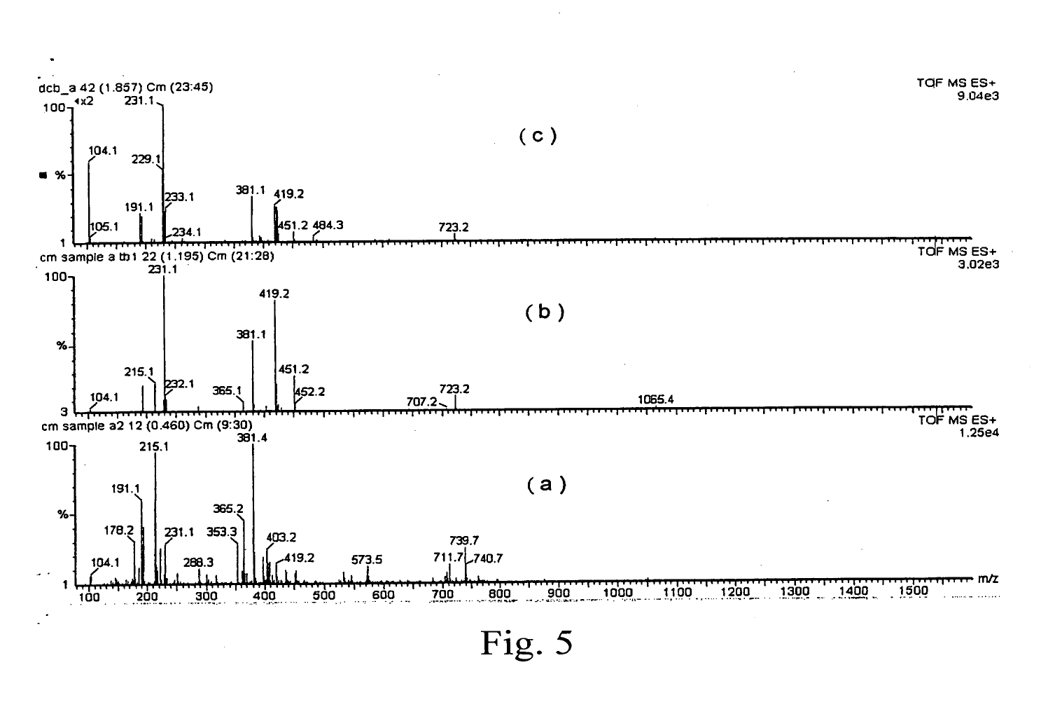

The present embodiment relates to treating a Rhizoma Chuanxiong sample by using the methanol extract method of the above-mentioned preparation of sample 1 to obtain a Rhizoma Chuanxiong extract; then performing a mass spectrum analysis at site A with the above ESI-MS analytic method; placing the extract at 4° C. after analyzing for 50 days; then taking the extract out and performing a mass spectrum analysis by using a mass spectrometer of the same type (but not the same one) at site A and B, respectively. The obtained spectra are shown in FIGS. 5(a), (b), and (c). The main mass spectrum peaks (m/z) in FIG. 5(a) are 178, 191, 193, 215, 231, 288, 353, 365, 381, 403, 419, 453, 533, 573, 711, 739, and 740, respectively. The main mass spectrum peaks in FIG. 5(b) are almost the same as those of FIG. 5(a), but the relative density of the mass spectrum peaks is somewhat different, such as m/z=381, 419. In FIG. 5(b), m/z=723 becomes stronger, but m/z=711 and 739 have no mass spectrum peak. It is also the same in FIG. 5(c).

### Embodiment 6

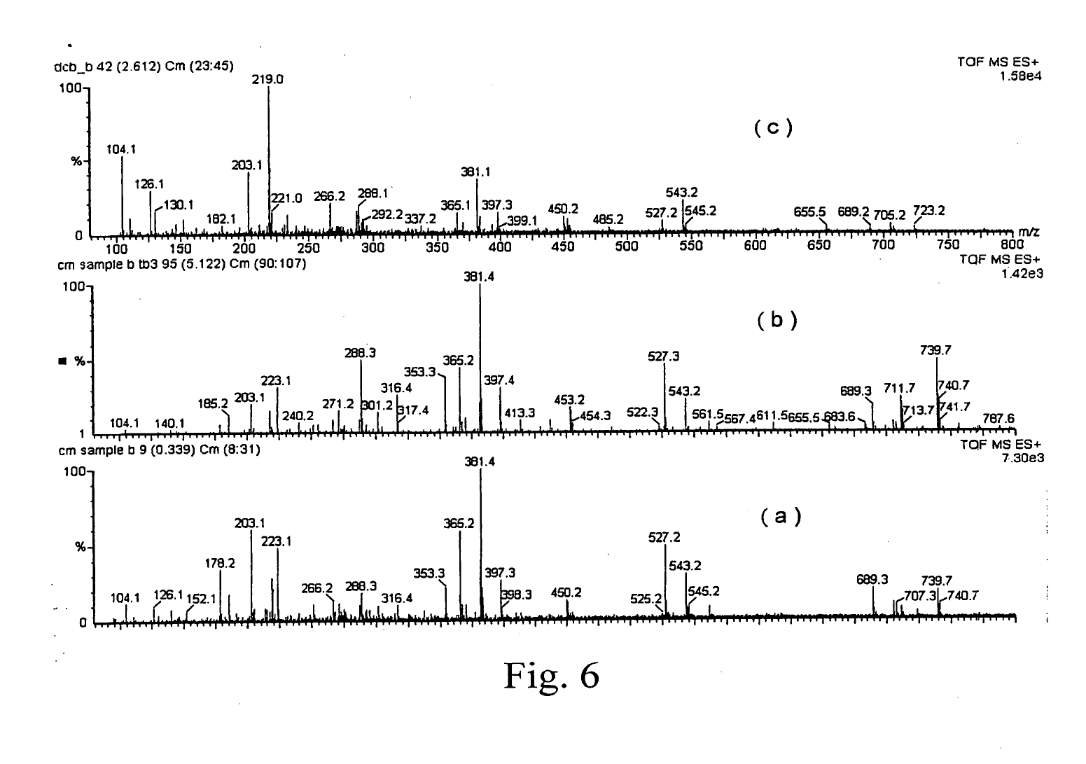

The present embodiment relates to a Radix Rehmanniae Preparata sample, and the experimental steps are analogous to Embodiment 5. The obtained spectra are shown in FIGS. 6(a), (b), and (c). The main mass spectrum peaks (m/z) in FIG. 6(a) are 126, 178, 185, 203, 219, 223, 266, 271, 288, 316, 353, 365, 381, 397, 527, 543, 689, and 739, respectively. The main mass spectrum peaks in FIG. 6(b) are almost the same as those of FIG. 6(a), but the relative denstiy of the mass spectrum peaks is somewhat different, such as m/z=178 and 185, 203, and 223. Compared with FIG. 6(a), in FIG. 6(c), the difference in the relative density between the mass spectrum peaks is larger, but the main mass spectrum peaks in FIG. 6(c) are substantially the same as those of FIG. 6(a).

### Embodiment 7

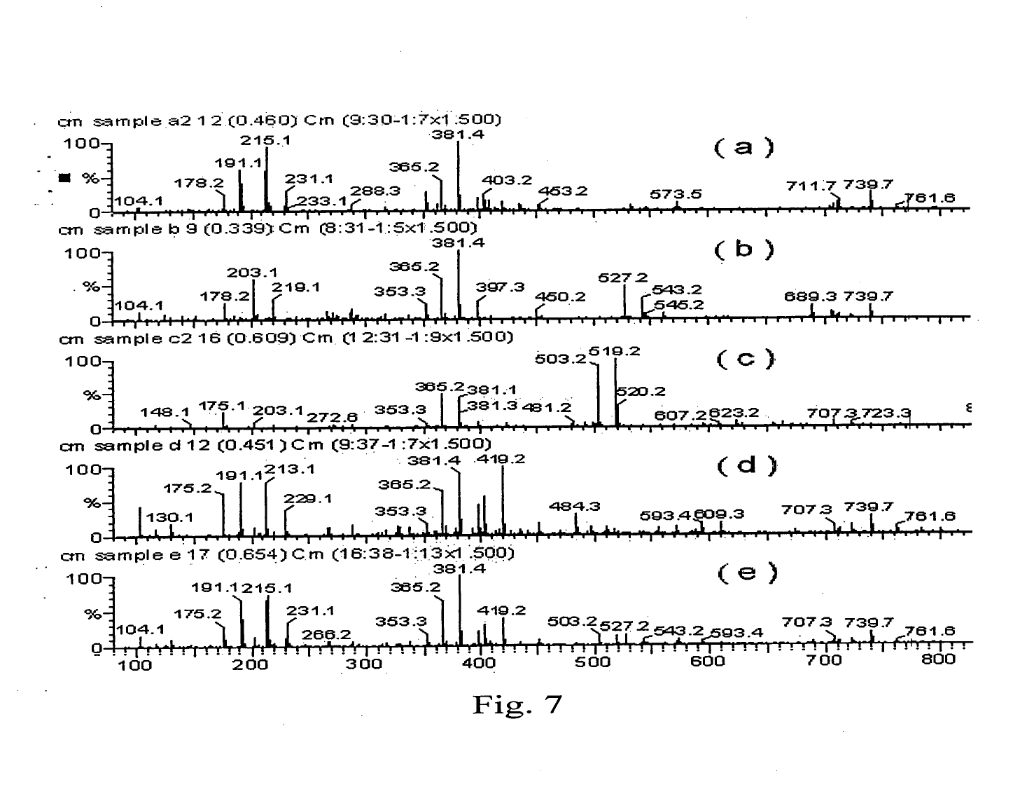

The present embodiment relates to treating four single medicines of Si Wu Tang by using the methanol extract method of the above-mentioned preparation of sample 1 to obtain the extracts of Rhizoma Chuanxiong, Radix Rehmanniae Preparata, Radix Paeoniae Alba, and Radix Angelicae Sinensis; then treating these four extracts by using the mix extract method of the above-mentioned preparation of sample 4; performing mass spectrum analysis for these sample extracts with the above ESI-MS analytic method. The obtained spectra are shown in FIGS. 7(a), (b), (c), (d), and (e). The main mass spectrum peaks (m/z) in FIG. 7(a) are 178, 191, 215, 231, 353, 365, 381, 403, and 739, respectively. The main mass spectrum peaks (m/z) in FIG. 7(b) are 178, 203, 219, 353, 365, 381, 397, 450, 527, 543, and 739, respectively. The main mass spectrum peaks (m/z) in FIG. 7(c) are 175, 353, 365, 381, 503, 519, and 707, respectively. The main mass spectrum peaks (m/z) in FIG. 7(d) are 175, 191, 213, 229, 353, 365, 381, 397, 403, 419, 484, 707, and 739, respectively. The main mass spectrum peaks (m/z) in FIG. 7(e) are 175, 191, 203, 213, 215, 231, 353, 365, 381, 397, 403, 419, 503, 519, 527, 543, 707 and 739, respectively.

### Embodiment 8

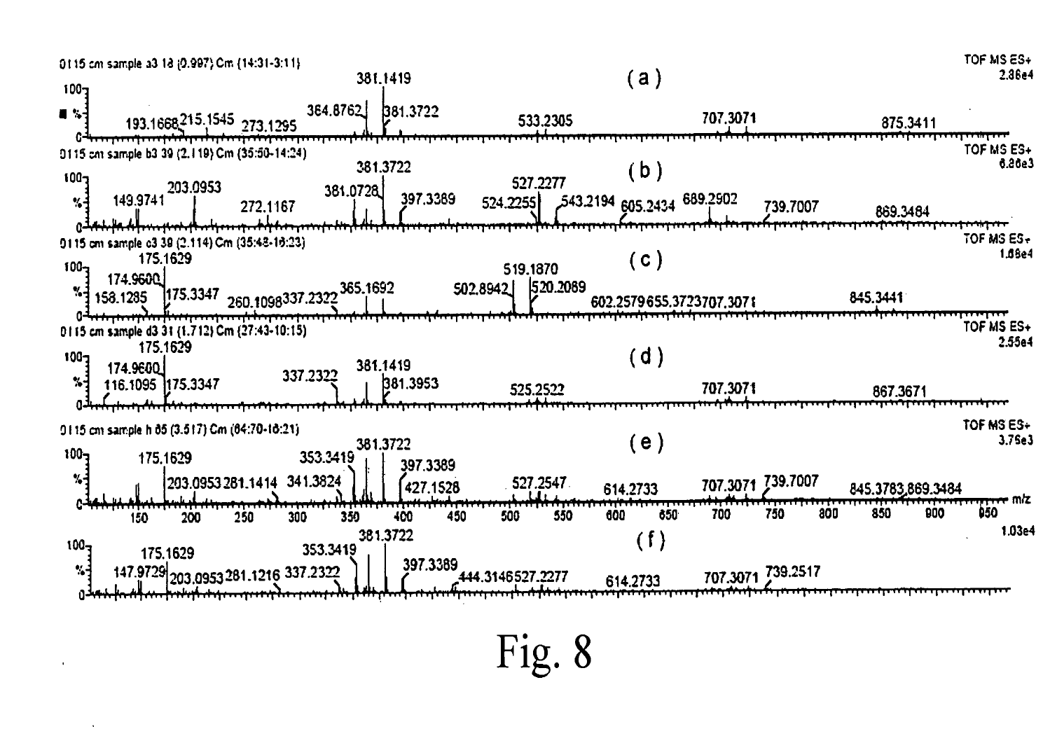

The present embodiment relates to treating four single medicines of Si Wu Tang by using the water extract method of the above-mentioned preparation of sample 2 to obtain the extracts of Rhizoma Chuanxiong, Radix Rehmanniae Preparata, Radix Paeoniae Alba, and Radix Angelicae Sinensis; then treating these four extracts by using the mix extract method of the above-mentioned preparation of sample 4; additionally mixing individual medicine of the same weight and extracting with water, to obtain the extract of Si Wu Tang; then performing mass spectrum analysis of these sample extracts with the above ESI-MS analytic method. The obtained spectra are shown in FIGS. 8(a), (b), (c), (d), (e), and (f). The main mass spectrum (m/z) peaks in FIG. 8(a) are 193, 215, 365, 381, 533, and 707, respectively. The main mass spectrum peaks (m/z) in FIG. 8(b) are 148, 150, 203, 272, 353, 365, 381, 397, 444, 527, 543, and 689, respectively. The main mass spectrum peaks (m/z) in FIG. 8(c) are 175, 365, 381, 503, and 519, respectively. The main mass spectrum peaks (m/z) in FIG. 8(d) are 175, 337, 365, 381, and 707, respectively. The main mass spectrum peaks (m/z) in FIG. 8(e) are 150, 175, 203, 337, 341, 353, 365, 381, 397, 502, 519, 527, and 707, respectively. The main mass spectrum peaks (m/z) in FIG. 8(f) are 150, 175, 203, 337, 344, 353, 365, 381, 397, 444, 503, 519, 527, and 707, respectively.

### Embodiment 9

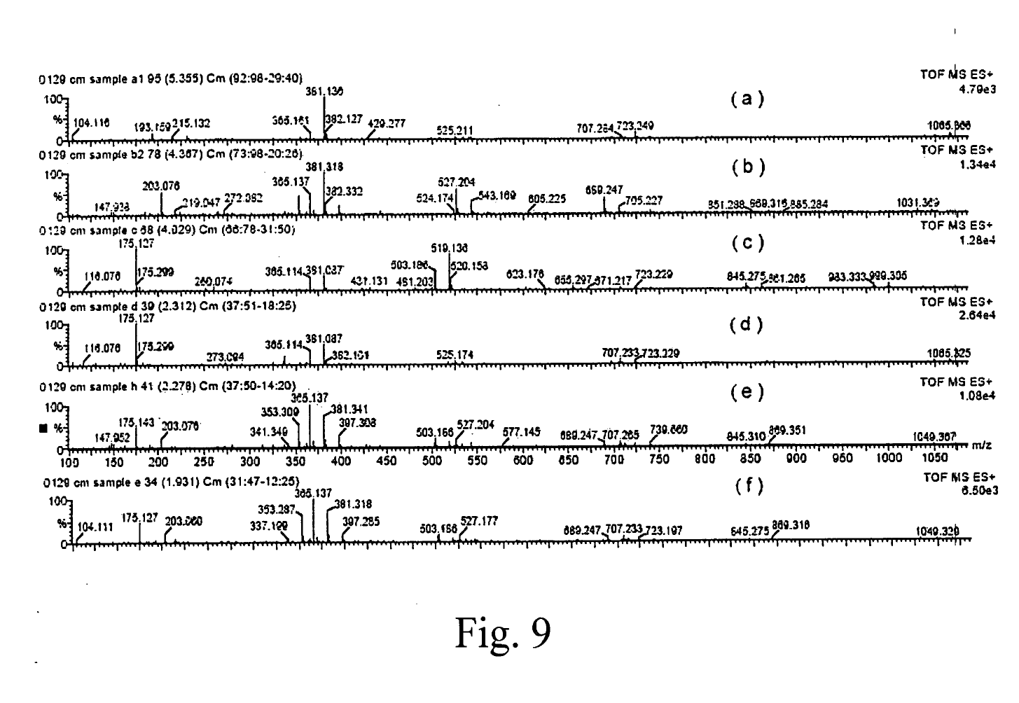

The present embodiment relates to placing the same extracts as Embodiment 8 at 4° C. for 14 days, then taking the extracts out and performing the mass spectrum analysis with the above ESI-MS analytic method. The obtained spectra are shown in FIGS. 9(a), (b), (c), (d), (e), and (f). The main mass spectrum peaks in the spectrum of Embodiment 9 are extremely similar to those of Embodiment 8, except that the relative density of some mass spectrum peaks is different.

### Embodiment 10

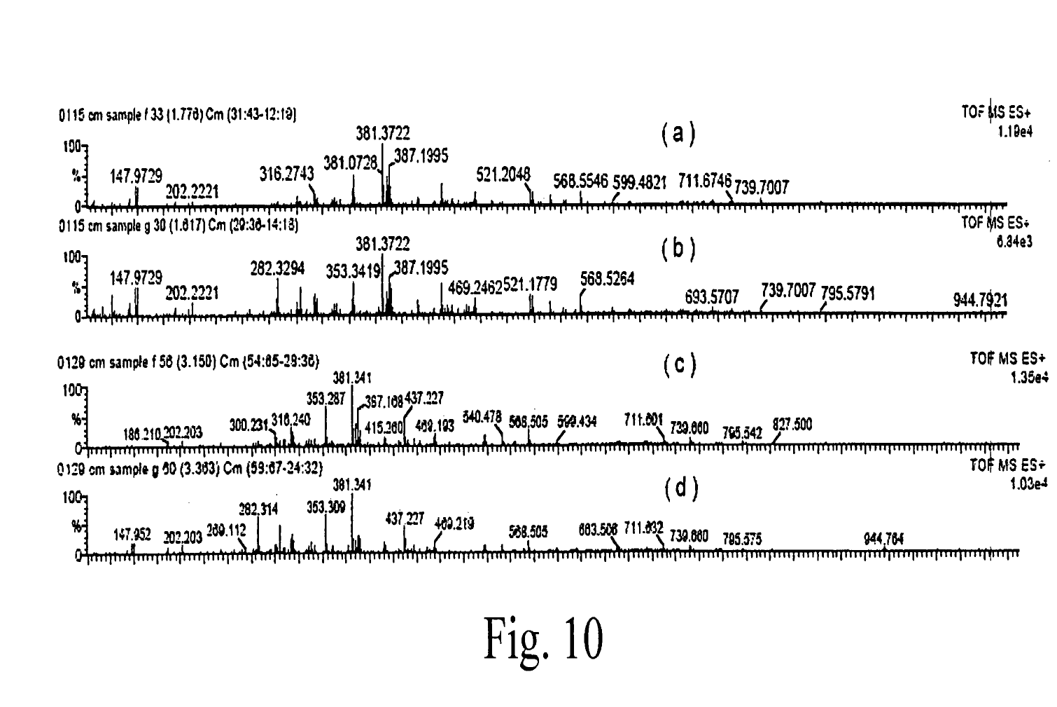

The present embodiment relates to treating Astragalus Membranaceus from different places of origin (such as Shanxi and Neimeng) by using the diethyl ether extract method of the above-mentioned preparation of sample 3 to obtain the extracts of Astragalus Membranaceus from Shanxi and Neimeng; then performing mass spectrum analysis with the above ESI-MS analytic method (the obtained spectra are shown in FIGS. 10(a) and (b)); placing these extracts at 4° C. for 14 days, then taking them out and performing the mass spectrum analysis (the obtained spectra are shown in FIGS. 10(c) and (d)). The main mass spectrum peaks (m/z) in FIG. 10(a) are 147, 202, 316, 353, 381, 387, 437, 469, 521, and 568, respectively. The main mass spectrum peaks (m/z) in FIG. 10(b) are substantially the same as those of FIG. 10(c), except that the main mass spectrum peaks in FIG. 10(b) have, apparently, two additional mass spectrum peaks, m/z=282 and 303. The main mass spectrum peaks (m/z) in FIGS. 10(c) and (d) are extremely similar to those of FIGS. 10(a) and (b), respectively.

In conclusion, the characteristic mass spectral fingerprint setting method of the present invention can accurately establish the characteristic mass spectral fingerprint database of Chinese herbal medicines and prescriptions, and can achieve the purpose of rapidly identifying unknown Chinese herbal medicines or prescriptions by further comparing the mass spectrum analysis and the database. The result shows that a good identification effect can be achieved by the method of the present invention for extracts of Chinese herbal medicines or Chinese herbal prescriptions.

1. A characteristic mass spectral fingerprint setting method for Chinese herbal medicines or prescriptions, which comprises the following steps:
(a) Extracting a Chinese herbal medicine or prescription with a solvent to obtain an analysis sample,
(b) Injecting the analysis sample via an autosampler of a liquid chromatograph and pushing a menstruum through a hydraulic pump, such that the analysis sample can be directly fed into a mass spectrometer with an electrospray ionization interface without going through a chromatographic separation column, to perform a mass spectrum analysis and obtain the mass spectrum data of the analysis sample, and
(c) Analyzing the obtained mass spectrum data, taking the mass spectrum peaks with a certain mass spectrum density and representing the features of the analysis sample as the selecting standard, and selecting the characteristic mass spectral fingerprint of the analysis sample.

2. A method according to claim 1, wherein the obtained characteristic mass spectral fingerprints of the analysis samples of various Chinese herbal medicines or prescriptions can be collected to establish a characteristic mass spectral fingerprint database.

3. A rapid identification method for Chinese herbal medicines or prescriptions, which comprises the following steps:
(a) Extracting a Chinese herbal medicine or prescription with a solvent to obtain an analysis sample,
(b) Injecting the analysis sample via an autosampler of a liquid chromatograph and pushing a menstruum through a hydraulic pump, such that the analysis sample can be directly fed into a mass spectrometer with an electrospray ionization interface without going through a chromatographic separation column, to perform a mass spectrum analysis and obtain the mass spectrum data of the analysis sample,
(c) Analyzing the obtained mass spectrum data, taking the mass spectrum peaks with a certain mass spectrum density and representing the features of the analysis sample as the selecting standard, and selecting the characteristic mass spectral fingerprint of the analysis sample.
(d) Repeating steps (a) to (c) to obtain the characteristic mass spectral fingerprints of various analysis samples so as to establish a characteristic mass spectral fingerprint database, and
(e) Directly feeding the analysis sample of a Chinese herbal medicine or prescription to be tested into a mass spectrometer with an electrospray ionization interface without going through a chromatographic separation column to perform a mass spectrum analysis, and comparing the obtained mass spectrum data with the characteristic mass spectral fingerprint database to rapidly identify the Chinese herbal medicine or prescription to be tested.

4. A method according to claim 1, wherein said solvent comprises water, methanol, or diethyl ether.

5. A method according to claim 1, wherein said liquid chromatograph is a high-performance liquid chromatograph.

6. A method according to claim 3, wherein said solvent comprises water, methanol, or diethyl ether.

7. A method according to claim 3, wherein said liquid chromatograph is a high-performance liquid chromatograph.
